SEQUENCE LISTING (19 sequences)

SEQ ID NO: 1 - protein, 452 aa, Artificial Sequence
  Synthetic Construct
QVQLVQSGAEVKKPGASVKVSCKASGYEFTSYWIHWVRQAPGQGLEWMGNISPNSGSANYNEKFKSRVTMTRDTSTSTVYMELSSLRSEDTAVYYCAREGPYSYYPSREYYGSDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 2 - protein, 126 aa, ARTIFICIAL SEQUENCE
  Synthetic Construct
QVQLVQSGAEVKKPGASVKVSCKASGYEFTSYWIHWVRQAPGQGLEWMGNISPNSGSANYNEKFKSRVTMTRDTSTSTVYMELSSLRSEDTAVYYCAREGPYSYYPSREYYGSDLWGQGTLVTVSS

SEQ ID NO: 3 - protein, 214 aa, ARTIFICIAL SEQUENCE
  Synthetic Construct
EIVLTQSPATLSLSPGERATLSCRASQSISNNLHWYQQKPGQAPRLLIYYTSRSVSGIPARFSGSGSGTDFTLTISSLEPEDFAVYYCGQNNEWPEVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 107 aa, ARTIFICIAL SEQUENCE
  Synthetic Construct
EIVLTQSPATLSLSPGERATLSCRASQSISNNLHWYQQKPGQAPRLLIYYTSRSVSGIPARFSGSGSGTDFTLTISSLEPEDFAVYYCGQNNEWPEVFGGGTKVEIK

SEQ ID NO: 5 - DNA, 1356 bp, ARTIFICIAL SEQUENCE
  Synthetic Construct
caggtgcagctggtgcagtctggtgctgaagtgaagaagcctggggcctcagtgaaggtgtcctgcaaggcatctggctacgagttcaccagctactggattcactgggtgcgacaggcccctggacaagggcttgagtggatgggaaatatttctcctaatagtggtagtgctaactacaatgagaagttcaagagcagagtcaccatgaccagggacacgtccacgagcacagtctacatggagctgagcagcctgagatctgaggacacggccgtgtattactgtgcgagagagggcccttacagttattatccgagtagggagtactatggctctgacctctgggggcaagggaccctagtcacagtctcctcagcctccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 6 - DNA, 642 bp, ARTIFICIAL SEQUENCE
  Synthetic Construct
gaaattgtgttgacacagtctccagccaccctgtctttgtctccaggggaaagagccaccctctcctgcagggccagtcaaagtatcagcaataacctacactggtaccaacagaaacctggccaggctcccaggctcctcatctattatacttcccggtccgtctctggcatcccagccaggttcagtggcagtgggtctgggacagacttcactctcaccatcagcagcctagagcctgaagattttgcagtttattactgtggacagaataacgagtggcctgaggtgttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 7 - protein, 11 aa, ARTIFICIAL SEQUENCE
  Synthetic Construct
RASQSISNNLH

SEQ ID NO: 8 - protein, 7 aa, ARTIFICIAL SEQUENCE
  Synthetic Construct
YTSRSVS

SEQ ID NO: 9 - protein, 9 aa, ARTIFICIAL SEQUENCE
  Synthetic Construct
GQNNEWPEV

SEQ ID NO: 10 - protein, 10 aa, ARTIFICIAL SEQUENCE
  Synthetic Construct
GYEFTSYWIH

SEQ ID NO: 11 - protein, 17 aa, ARTIFICIAL SEQUENCE
  Synthetic Construct
NISPNSGSANYNEKFKS

SEQ ID NO: 12 - protein, 17 aa, ARTIFICIAL SEQUENCE
  Synthetic Construct
EGPYSYYPSREYYGSDL

SEQ ID NO: 13 - protein, 73 aa, Homo sapiens
ASVATELRCQCLQTLQGIHPKNIQSVNVKSPGPHCAQTEVIATLKNGRKACLNPASPIVKKIIEKMLNSDKSN

SEQ ID NO: 14 - protein, 73 aa, Homo sapiens
APLATELRCQCLQTLQGIHLKNIQSVKVKSPGPHCAQTEVIATLKNGQKACLNPASPMVKKIIEKMLKNGKSN

SEQ ID NO: 15 - protein, 73 aa, Homo sapiens
ASVVTELRCQCLQTLQGIHLKNIQSVNVRSPGPHCAQTEVIATLKNGKKACLNPASPMVQKIIEKILNKGSTN

SEQ ID NO: 16 - protein, 74 aa, Homo sapiens
AAVLRELRCVCLQTTQGVHPKMISNLQVFAIGPQCSKVEVVASLKNGKEICLDPEAPFLKKVIQKILDGGNKEN

SEQ ID NO: 17 - protein, 75 aa, Homo sapiens
VSAVLTELRCTCLRVTLRVNPKTIGKLQVFPAGPQCSKVEVVASLKNGKQVCLDPEAPFLKKVIQKILDSGNKKN

SEQ ID NO: 18 - protein, 70 aa, Homo sapiens
AELRCMCIKTTSGIHPKNIQSLEVIGKGTHCNQVEVIATLKDGRKICLDPDAPRIKKIVQKKLAGDESAD

SEQ ID NO: 19 - protein, 72 aa, Homo sapiens
SAKELRCQCIKTYSKPFHPKFIKELRVIESGPHCANTEIIVKLSDGRELCLDPKENWVQRVVEKFLKRAENS

(19) United States
(12) Patent Application Publication
(10) Pub. No.: US 2023/0159630 A1
(43) Pub. Date: May 25, 2023

(54) PAN-ELR+ CXC CHEMOKINE ANTIBODIES FOR THE TREATMENT OF RESPIRATORY DISEASE

(71) Applicant: Eli Lilly and Company, Indianapolis, IN (US)

(72) Inventors: Dipak R Patel, San Diego, CA (US); Derrick Ryan Witcher, Fishers, IN (US); Michael Edward Woodman, Fishers, IN (US)

(21) Appl. No.: 17/920,118

(22) PCT Filed: Apr. 16, 2021
(86) PCT No.: PCT/US2021/027776

§ 371 Date: Oct. 20, 2022

Related U.S. Application Data
(60) Provisional application No. 63/015,308, filed on Apr. 24, 2020.

(51) Int. Cl. C07K 16/24; A61P 11/00; A61P 31/14

(52) U.S. Cl. CPC C07K 16/24; A61P 11/00; A61P 31/14; A61K 2039/505

(57) ABSTRACT

Provided herein are methods and uses of antibodies that bind all seven human ELR+CXC chemokines with high affinity for preventing and/or treating respiratory diseases, e.g., acute respiratory distress syndrome (ARDS). The present invention also relates to doses and dosing regimens for the methods and uses of antibodies that bind all seven human ELR+CXC chemokines with high affinity for preventing and/or treating respiratory diseases, e.g., ARDS.

The present invention relates to methods and uses of antibodies against ELR+CXC chemokines for preventing and/or treating respiratory diseases, e.g., acute respiratory distress syndrome (ARDS). The present invention also relates to doses and dosing regimens for the methods and uses of antibodies against ELR+CXC chemokines for preventing and/or treating respiratory diseases, e.g., ARDS.

ELR+CXC chemokines (so-called because members of the chemokine family all possess an E-L-R amino acid motif immediately adjacent to their CXC motif) play an important role in a variety of pathogenic mechanisms, including the migration of neutrophils to sites of inflammation and angiogenesis. Neutrophils contribute to the pathogenesis of several acute and chronic inflammatory and autoimmune diseases.

Chemokines are grouped into four subfamilies: CXC, CC, (X)C, and CX3C. In the CXC chemokines, one amino acid separates the first two cysteines (“the CXC motif”). ELR+CXC chemokines are ligands for CXCR 1 and/or CXCR2 chemokine receptors, which are G-protein coupled seven transmembrane domain-type receptors that specifically bind ELR+CXC chemokines. The seven human ELR+CXC chemokines are human growth-regulated oncogene (“Gro”)-alpha (also known as CXCL1), human Gro-beta (also known as CXCL2), human Gro-gamma (also known as CXCL3), human ENA-78 (also known as CXCL5 or human epithelial neutrophil activating peptide-78), human GCP-2 (also known as CXCL6 or human granulocyte chemotactic protein-2), human NAP-2 (also known as CXCL7 or human neutrophil activating protein-2), and human IL-8 (also known as CXCL8 or human interleukin-8). All ELR+CXC chemokines bind the CXCR2 receptor; moreover, some ELR+CXC chemokines bind both CXCR 1 and CXCR2 receptors (i.e., CXCL6 and CXCL8), all of which contributes to redundancy in the activation pathways. Neutralizing all seven ELR+CXC chemokines could impact the ability of CXCR1+ or CXCR2+ cells to migrate to sites of inflammation.

Antibodies that bind and neutralize all seven human ELR+CXC chemokines have been previously described, e.g., in WO 2014149733, EP 2970447B1, U.S. Pat. No. 9,290,570. Given their ability to bind and neutralize all seven human ELR+CXC chemokines, those antibodies offer advantages over monotherapies targeting single human ELR+CXC chemokines and combination therapies targeting multiple human ELR+CXC chemokines. One of such antibodies that bind all seven human ELR+CXC chemokines is Antibody 1 that comprises light chain complementarity determining regions (“LCDR”) LCDR1, LCDR2, LCDR3, and heavy chain complementarity determining regions (“HCDR”) HCDR1, HCDR2, HCDR3, wherein LCDR1 comprises SEQ ID NO: 7, LCDR2 comprises SEQ ID NO: 8, LCDR3 comprises SEQ ID NO: 9, HCDR1 comprises SEQ ID NO: 10, HCDR2 comprises SEQ ID NO: 11, and HCDR3 comprises SEQ ID NO: 12. Antibody 1 comprises a heavy chain variable region comprising the amino acid sequence of SEQ ID NO: 2 and a light chain variable region comprising the amino acid sequence of SEQ ID NO: 4. Antibody 1 comprises a heavy chain having the amino acid sequence of SEQ ID NO: 1 and a light chain having the amino acid sequence of SEQ ID NO: 3. It was shown that Antibody 1 binds an epitope that is common to all seven human ELR+CXC chemokines and neutralize the activities of all seven human ELR+CXC chemokines. By binding to all seven ELR+CXC chemokines, both the CXCR1 and the CXCR2 pathways can be blocked, which may result in more effective inhibition of neutrophil trafficking.

ARDS is a life-threatening respiratory disease characterized by inflammation of the lungs, which can be widespread and rapid in onset. ARDS is caused by damage to the alveolar epithelial and endothelial barriers, leading to accumulation of fluid and innate inflammatory cells that trigger further inflammation and tissue injury. This culminates in pulmonary edema and progressive pulmonary failure/death. ARDS has a mortality rate reported as high as 30-40%. Symptoms associated with ARDS include shortness of breath, rapid breathing and bluish skin coloration, in association with disease or injury. Formal diagnosis of ARDS is challenging as scientific and medical definitions have evolved. One definition, known as the “Berlin definition”, relies on radiological imagining of lung and PaO2/FiO2 ratios (Ranieri et al, 2012, JAMA, 307 (23): 2526-33). According to the Berlin definition, ARDS is characterized according to the following factors: acute onset respiratory symptoms following lung insult; unexplained bilateral opacities on chest imaging; respiratory failure (not explained by heart failure or volume overload); decreased PaO2/FiO2 ratio. The Berlin definition also allows for staging of ARDS according to: mild ARDS: 201-300 mmHg (≤39.9 kPa); moderate ARDS. 101-200 mmHg (≤26.6 kPa); and severe ARDS: ≤100 mmHg (≤13.3 kPa). However, even in those who recover, although lung function may gradually improve over a period of six months to a year, patients may be left with significant scarring and lower than normal lung volumes.

There are presently no approved treatments for preventing and/or treating ARDS. A complicating factor in developing a therapy for ARDS is that ARDS and associated mortalities develop from a number of diseases and injuries, including interstitial lung disease, viral insult such as coronavirus disease 2019 (i.e., COVID-19), caused by SARS-CoV-2 virus, and middle eastern respiratory syndrome (MERS), and therapy induced insult such as CAR-T therapy induced ARDS. Additionally, ARDS involves a number of molecular pathways involving numerous immune and epithelial targets. Further, complex dysregulation of the body's own immune response, following disease or injury (for example, as in COVID-19), leading to a phenomenon known as cytokine storm has also been associated with ARDS. As such, there remains an urgent and unmet need for the prevention and/or treatment of ARDS.

The present disclosure provides methods and uses for the prevention and/or treatment of ARDS. In one aspect, provided herein are methods of preventing and/or treating ARDS in a human patient in need thereof by administering to the human patient a therapeutically effective amount of an antibody that binds all seven human ELR+CXC chemokines, e.g., Antibody 1, or a pharmaceutical composition comprising such an antibody. In some embodiments, provided herein are methods of preventing and/or treating ARDS in a human patient in need thereof comprising administering to the human patient a therapeutically effective amount of an antibody that binds human growth-regulated oncogene (“Gro”)-alpha, human Gro-beta, human Gro-gamma, human epithelial neutrophil activating peptide-78, human granulocyte chemotactic protein-2, human neutrophil activating protein-2, and human interleukin-8, or a pharmaceutical composition comprising such an antibody, wherein the antibody comprises light chain complementarity determining regions (“LCDR”) LCDR1, LCDR2, LCDR3, and heavy chain complementarity determining regions (“HCDR”) HCDR1, HCDR2, HCDR3, wherein LCDR1 comprises SEQ ID NO: 7, LCDR2 comprises SEQ ID NO: 8, LCDR3 comprises SEQ ID NO: 9, HCDR1 comprises SEQ ID NO: 10, HCDR2 comprises SEQ ID NO: 11, and HCDR3 comprises SEQ ID NO: 12. In some embodiments, the antibody comprises a heavy chain variable region comprising the amino acid sequence of SEQ ID NO: 2 and a light chain variable region comprising the amino acid sequence of SEQ ID NO: 4. In some embodiments, the antibody comprises a heavy chain having the amino acid sequence of SEQ ID NO: 1 and a light chain having the amino acid sequence of SEQ ID NO: 3.

IL-8 is highly correlated with adverse outcomes in ARDS. IL-8 and other ELR+ chemokines attract neutrophils into the injured alveolus. Once neutrophils enter the inflamed lung, they secrete proteases, reactive oxygen species, neutrophil extracellular traps (NETs), and other pro-inflammatory mediators that cause tissue injury and further contribute to inflammation. Neutrophil NETs could uniquely contribute to ARDS by inducing IL-ab (cytokine storm), M1 alveolar macrophage polarization, mucosal secretions, and microthrombi. Multiple ELR+ chemokines are elevated in ARDS patients in general and COVID-19 patients in particular (plasma and bronchoalveolar fluid [BALF]) and IL-8 is elevated in patients infected with the related coronaviruses that cause SARS and MERS (plasma and BALF). The ELR+ chemokines that bind to CXCR1 and CXCR2 are involved in angiogenesis and neutrophil migration. Neutralizing all seven CXCR1/2 ligands can potentially decrease mortality in ARDS, in general, and ARDS associated with coronaviral infections (SARS, MERS, COVID-19, etc.) in particular, by blocking neutrophil trafficking to the lung, decreasing multiple neutrophil-mediated contributions to this disease. In contrast to other molecules that target individual ligands or receptors in the CXCR1/2 network, Antibody 1 is uniquely able to bind to and neutralize all seven CXCR1/2 ligands.

In some embodiments, a method of preventing ARDS in a patient is provided, comprising administering to said patient a therapeutically effective amount of an antibody that binds all seven human ELR+CXC chemokines, e.g., Antibody 1, or a pharmaceutical composition comprising such an antibody. In some embodiments, the patient is at risk of developing ARDS. In some embodiments, the patient has a respiratory insult. In some embodiments, the respiratory insult is a respiratory disease. According to some embodiments, the respiratory insult is a respiratory injury.

In some embodiments, a method of treating ARDS in a patient is provided comprising administering to said patient a therapeutically effective amount of an antibody that binds all seven human ELR+CXC chemokines, e.g., Antibody 1, or a pharmaceutical composition comprising such an antibody. In some embodiments, the patient is diagnosed as having mild ARDS. In some embodiments, the patient is diagnosed as having moderate ARDS. In some embodiments, the patient is diagnosed as having severe ARDS. In some embodiments, the patient is diagnosed as having one of mild, moderate or severe ARDS according to the Berlin definition.

According to some embodiments of the methods provided herein, the patient has a viral infection. In some embodiments, the viral infection is a coronavirus infection. In some embodiments, the coronavirus is SARS-CoV-2. According to some embodiments of the methods provided herein, the patient has COVID-19. According to some embodiments of the methods provided herein, the patient has pneumonia. According to some embodiments of the methods provided herein, the patient has asthma. According to some embodiments of the methods provided herein, the patient has chronic obstructive pulmonary disease (COPD). According to some embodiments of the methods provided herein, the patient has pulmonary fibrosis. According to some embodiments of the methods provided herein, the patient has interstitial lung disease.

Also provided herein are antibodies that bind all seven human ELR+CXC chemokines, e.g., Antibody 1, or a pharmaceutical composition comprising such an antibody, for use in the prevention and/or treatment of ARDS. In some embodiments, provided herein is an antibody that binds human growth-regulated oncogene (“Gro”)-alpha, human Gro-beta, human Gro-gamma, human epithelial neutrophil activating peptide-78, human granulocyte chemotactic protein-2, human neutrophil activating protein-2, and human interleukin-8, wherein the antibody comprises light chain complementarity determining regions (“LCDR”) LCDR1, LCDR2, LCDR3 and heavy chain complementarity determining regions (“HCDR”) HCDR1, HCDR2, HCDR3, wherein LCDR1 comprises SEQ ID NO: 7, LCDR2 comprises SEQ ID NO: 8, LCDR3 comprises SEQ ID NO: 9, HCDR1 comprises SEQ ID NO: 10, HCDR2 comprises SEQ ID NO: 11, and HCDR3 comprises SEQ ID NO: 12, or a pharmaceutical composition comprising such an antibody, for use in the prevention and/or treatment of ARDS. In some embodiments, the antibody comprises a heavy chain variable region comprising the amino acid sequence of SEQ ID NO: 2 and a light chain variable region comprising the amino acid sequence of SEQ ID NO: 4. In some embodiments, the antibody comprises a heavy chain having the amino acid sequence of SEQ ID NO: 1 and a light chain having the amino acid sequence of SEQ ID NO: 3.

Also provided herein are uses of antibodies that bind all seven human ELR+CXC chemokines, e.g., Antibody 1, in the manufacture of a medicament for in the prevention and/or treatment of ARDS. In some embodiments, provided herein is use of an antibody that binds human growth-regulated oncogene (“Gro”)-alpha, human Gro-beta, human Gro-gamma, human epithelial neutrophil activating peptide-78, human granulocyte chemotactic protein-2, human neutrophil activating protein-2, and human interleukin-8, wherein the antibody comprises light chain complementarity determining regions (“LCDR”) LCDR1, LCDR2, LCDR3 and heavy chain complementarity determining regions (“HCDR”) HCDR1, HCDR2, HCDR3, wherein LCDR1 comprises SEQ ID NO: 7, LCDR2 comprises SEQ ID NO: 8, LCDR3 comprises SEQ ID NO: 9, HCDR1 comprises SEQ ID NO: 10, HCDR2 comprises SEQ ID NO: 11, and HCDR3 comprises SEQ ID NO: 12, in the manufacture of a medicament for in the prevention and/or treatment of ARDS. In some embodiments, the antibody comprises a heavy chain variable region comprising the amino acid sequence of SEQ ID NO: 2 and a light chain variable region comprising the amino acid sequence of SEQ ID NO: 4. In some embodiments, the antibody comprises a heavy chain having the amino acid sequence of SEQ ID NO: 1 and a light chain having the amino acid sequence of SEQ ID NO: 3.

In some embodiments, the antibody that binds all seven human ELR+CXC chemokines, e.g., Antibody 1, or a pharmaceutical composition comprising such an antibody, is administered intravenously. In some embodiments, the antibody that binds all seven human ELR+CXC chemokines, e.g., Antibody 1, or a pharmaceutical composition comprising such an antibody, is administered subcutaneously.

In some embodiments, the antibody that binds all seven human ELR+CXC chemokines, e.g., Antibody 1, or a pharmaceutical composition comprising such an antibody, is administered to the patient for two to three doses. In some embodiments, the antibody that binds all seven human ELR+CXC chemokines, e.g., Antibody 1, or a pharmaceutical composition comprising such an antibody, is administered intravenously to the patient for two to three doses.

In some embodiments, the antibody that binds all seven human ELR+CXC chemokines, e.g., Antibody 1, or a pharmaceutical composition comprising such an antibody, is administered intravenously at a dose of about 100 mg to about 1200 mg (e.g., about 100 mg to about 1200 mg, about 200 mg to about 1150 mg, about 300 mg to about 1100 mg, about 600 mg to about 1000 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 850 mg, about 900 mg, about 950 mg, about 1000 mg, about 1050 mg, about 1100 mg, about 1150 mg, or about 1200 mg).

As shown below, the bioavailability for subcutaneous injection of Antibody 1 is estimated to be approximately 65% of intravenous injection of Antibody 1. In some embodiments, the antibody that binds all seven human ELR+CXC chemokines, e.g., Antibody 1, or a pharmaceutical composition comprising such an antibody, is administered subcutaneously at a dose of about 150 mg to about 1800 mg (e.g., about 150 mg to about 1800 mg, about 200 mg to about 1700 mg, about 300 mg to about 1600 mg, about 400 mg to about 1500 mg, about 500 mg to about 1400 mg, about 600 mg to about 1300 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, or about 1800 mg).

As used herein, the term “human ELR+CXC chemokines” refers to the seven known CXC chemokines that have an E-L-R motif and that bind to CXCR 1 and/or CXCR2 receptor. The human ELR+CXC chemokines are human Gro-alpha (also known as CXCL1) (SEQ ID NO: 13), human Gro-beta (also known as CXCL2) (SEQ ID NO: 14), human Gro-gamma (also known as CXCL3) (SEQ ID NO: 15), human ENA-78 (also known as CXCL5) (SEQ ID NO: 16), human GCP-2 (also known as CXCL6) (SEQ ID NO: 17), human NAP-2 (also known as CXCL7) (SEQ ID NO: 18), and human IL-8 (also known as CXCL8) (SEQ ID NO: 19). Collectively, all seven human ELR+CXC chemokines are called “human pan-ELR+CXC chemokines” herein.

The term “antibody,” as used herein, refers to monoclonal immunoglobulin molecules comprising four polypeptide chains, two heavy (H) chains and two light (L) chains inter-connected by disulfide bonds. Each heavy chain comprises a heavy chain variable region (HCVR) and a heavy chain constant region. The heavy chain constant region comprises three domains, CH1, CH2, and CH3. Each light chain is comprised of a light chain variable region (LCVR) and a light chain constant region, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxyl-terminus in the following order: FR1, CDR1, FR2, CDR2, FR3, CDR3, FR4. The CDR regions in HCVR are termed HCDR1, HCDR2, and HCDR3. The CDR regions in LCVR are termed LCDR1, LCDR2, and LCDR3. The CDRs contain most of the residues which form specific interactions with the antigen. There are currently three systems of CDR assignments for antibodies that are commonly used for sequence delineation. The Kabat CDR definition (Kabat et al., “Sequences of Proteins of Immunological Interest,” National Institutes of Health, Bethesda, Md. (1991)) is based upon antibody sequence variability. The Chothia CDR definition (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)) is based on three-dimensional structures of antibodies and topologies of the CDR loops. The Chothia CDR definitions are identical to the Kabat CDR definitions with the exception of HCDR1 and HCDR2. For the purposes of the present invention, a hybrid of the Kabat and Chothia definitions are used to define CDRs. The assignment of amino acids in the HCVR and LCVR regions is in accordance with the Kabat numbering convention. It is further understood that the term “antibody” encompasses any cellular post-translational modifications to the antibody including, but not limited to, acylation and glycosylation.

As used herein, the term “septa-specific antibody” refers to an antibody that binds all seven human ELR+CXC chemokines with high affinity (e.g., with binding affinity (K_{D}) in the range of from about 5×10⁻¹¹ M to about 1×10⁻⁹ M).

As used herein, a “patient,” “individual,” “subject,” refers to a human with a disease, disorder, or condition that would benefit from a decreased level of human ELR+CXC chemokines or decreased bioactivity induced by human ELR+CXC chemokines.

As used herein, “prevention”, “prevent”, and/or “preventing”, which are used interchangeably herein, are intended to refer to all processes wherein there may be a slowing, interrupting, arresting, controlling, stopping, alleviating symptoms or complications or reversing of the progression of a respiratory disease, for example, caused by an injury, insult such as SARS-CoV-2 viral infection or disease such as COVID-19 disease, or therapy induced insult such as CAR-T therapy, whereby the respiratory disease does not progress to ARDS or does not progress to a more severe stage of ARDS, for example, as defined by the Berlin definition. As used herein, prevention is not intended to necessarily indicate a total elimination of all disorder symptoms.

As used interchangeably herein, “treatment” and/or “treating” and/or “treat” are intended to refer to all processes wherein there may be a slowing, interrupting, arresting, controlling, stopping, alleviating symptoms or complications or reversing of the progression of ARDS, but does not necessarily indicate a total elimination of all disorder symptoms.

As may be used herein, the terms “about” or “approximately”, when used in reference to a particular recited numerical value or range of values, means that the value may vary from the recited value by no more than 10% (e.g., +/−10%). For example, as used herein, the expression “about 100” includes 90 and 110 and all values in between (e.g., 91, 92, 93, 94, etc.).

### EXAMPLE 1: A SINGLE-ASCENDING DOSE STUDY TO EVALUATE THE SAFETY, TOLERABILITY, PHARMACOKINETICS, AND PHARMACODYNAMICS OF ANTIBODY 1 IN HEALTHY SUBJECTS (NCT02148627)

The primary objective of this study is to explore the safety and tolerability of a single dose of Antibody 1 in healthy subjects, including Japanese subjects, in order to define an appropriate dose range for further clinical research. The endpoints for this objective are incidence of SAEs (serious adverse events) and TEAEs (treatment-emergent adverse events). The secondary objective of this study is to characterize the pharmacokinetics (PK) of Antibody 1, including estimation of the bioavailability following subcutaneous (SC) administration of a single dose of Antibody 1, in healthy subjects, including Japanese subjects. The endpoints include Cmax (maximum observed drug concentration), tmax (time to reach Cmax), AUCs (area under the concentration-time curve during dosing interval at steady) and the presence of antidrug antibodies.

In this study, Antibody 1 has been administered to 39 healthy subjects in a single-ascending dose study in which doses of 10 mg, 30 mg, 100 mg, 200 mg, 400 mg, or 700 mg of Antibody 1 were administered as a slow intravenous (IV) infusion; or the dose of 100 mg of Antibody 1 was administered by SC injection to assess the bioavailability. In addition, skin blisters were induced in subjects who received IV doses of 10 mg, 30 mg, 100 mg, 200 mg, and 400 mg of Antibody 1 to assess neutrophil chemotaxis and accumulation.

There were no deaths or serious adverse events (SAEs) reported. Adverse events (AEs) reported were graded using the Common Terminology Criteria for Adverse Events (CTCAE). All AEs reported were Grade 1, except for 1 subject who reported a Grade 2 event, which the investigator considered to be unrelated to study drug.

There were no clinically significant changes in hematology (including peripheral blood neutrophil counts) or urinalysis.

There were no clinically significant changes in vital signs or electrocardiograms (ECG).

Antibody 1 exhibited linear PK in the dose range tested, with an estimated terminal half-life (t1/2) of approximately 2 weeks. The estimated bioavailability for SC injection of Antibody 1 is approximately 65% of IV injection.

The preliminary pharmacodynamic (PD) assessment was focused on the percentage of neutrophils present in the blister fluid. The neutrophil data were highly variable, but a trend of dose-dependent decreases of neutrophils in the blister fluid was observed.

### EXAMPLE 2: CLINICAL STUDY OF ANTIBODY 1 FOR PREVENTION AND/OR TREATMENT OF ARDS IN COVID-19 PATIENTS

A clinical study comparing prevention and/or treatment of ARDS in patients with COVID-19 with Antibody 1, can be undertaken as described herein. Briefly, patients positive for COVID-19 can be administered intravenously with 1200 mg of Antibody 1 or placebo for two to three doses on Day 0, Day 2 or 3, and Day 7. Day 7 dose will be administered only if the patient is in respiratory distress. Day 28 will be the primary endpoint. The primary endpoint can be all cause mortality at Day 28, or proportion of patients alive and respiratory failure free at Day 28. There will be 11 weeks of follow up time. The secondary endpoints can be number of days alive and ventilator-free; number of days in ICU; number of days in hospital; and/or number of days before patient returns to baseline oxygen requirement. Patients can be assessed for respiratory disease presence, ARDS presence, or respiratory disease or ARDS progression during and following treatment. Assessment may include chest imagining and PaO2/FiO2 ratio assessment.

### SEQUENCE LISTING

Antibody 1 Heavy chain amino acid sequence:
SEQ ID NO: 1
QVQLVQSGAEVKKPGASVKVSCKASGYEFTSYWIH
WVRQAPGQGLEWMGNISPNSGSANYNEKFKSRVTM
TRDTSTSTVYMELSSLRSEDTAVYYCAREGPYSYY
PSREYYGSDLWGQGTLVTVSSASTKGPSVFPLAPC
SRSTSESTAALGCLVKDYFPEPVTVSWNSGALTSG
VHTFPAVLQSSGLYSLSSVVTVPSSSLGTKTYTCN
VDHKPSNTKVDKRVESKYGPPCPPCPAPEFLGGPS
VFLFPPKPKDTLMISRTPEVTCVVVDVSQEDPEVQ
FNWYVDGVEVHNAKTKPREEQFNSTYRVVSVLTVL
HQDWLNGKEYKCKVSNKGLPSSIEKTISKAKGQPR
EPQVYTLPPSQEEMTKNQVSLTCLVKGFYPSDIAV
EWESNGQPENNYKTTPPVLDSDGSFFLYSRLTVDK
SRWQEGNVFSCSVMHEALHNHYTQKSLSLSLG
Antibody 1 Heavy chain variable region:
SEQ ID NO: 2
QVQLVQSGAEVKKPGASVKVSCKASGYEFTSYWIH
WVRQAPGQGLEWMGNISPNSGSANYNEKFKSRVTM
TRDTSTSTVYMELSSLRSEDTAVYYCAREGPYSY
YPSREYYGSDLWGQGTLVTVSS
Antibody 1 Light chain amino acid sequence:
SEQ ID NO: 3
EIVLTQSPATLSLSPGERATLSCRASQSISNNLHW
YQQKPGQAPRLLIYYTSRSVSGIPARFSGSGSGTD
FTLTISSLEPEDFAVYYCGQNNEWPEVFGGGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
Antibody 1 Light chain variable region:
SEQ ID NO: 4
EIVLTQSPATLSLSPGERATLSCRASQSISNNLHW
YQQKPGQAPRLLIYYTSRSVSGIPARFSGSGSGTD
FTLTISSLEPEDFAVYYCGQNNEWPEVFGGGTKVE
IK
Antibody 1 Heavy chain DNA sequence:
SEQ ID NO: 5
CAGGTGCAGCTGGTGCAGTCTGGTGCTGAAGTGAA
GAAGCCTGGGGCCTCAGTGAAGGTGTCCTGCAAGG
CATCTGGCTACGAGTTCACCAGCTACTGGATTCAC
TGGGTGCGACAGGCCCCTGGACAAGGGCTTGAGTG
GATGGGAAATATTTCTCCTAATAGTGGTAGTGCTA
ACTACAATGAGAAGTTCAAGAGCAGAGTCACCATG
ACCAGGGACACGTCCACGAGCACAGTCTACATGGA
GCTGAGCAGCCTGAGATCTGAGGACACGGCCGTGT
ATTACTGTGCGAGAGAGGGCCCTTACAGTTATTAT
CCGAGTAGGGAGTACTATGGCTCTGACCTCTGGGG
GCAAGGGACCCTAGTCACAGTCTCCTCAGCCTCCA
CCAAGGGCCCATCGGTCTTCCCGCTAGCGCCCTGC
TCCAGGAGCACCTCCGAGAGCACAGCCGCCCTGGG
CTGCCTGGTCAAGGACTACTTCCCCGAACCGGTGA
CGGTGTCGTGGAACTCAGGCGCCCTGACCAGCGGC
GTGCACACCTTCCCGGCTGTCCTACAGTCCTCAGG
ACTCTACTCCCTCAGCAGCGTGGTGACCGTGCCCT
CCAGCAGCTTGGGCACGAAGACCTACACCTGCAAC
GTAGATCACAAGCCCAGCAACACCAAGGTGGACAA
GAGAGTTGAGTCCAAATATGGTCCCCCATGCCCAC
CCTGCCCAGCACCTGAGTTCCTGGGGGGACCATCA
GTCTTCCTGTTCCCCCCAAAACCCAAGGACACTCT
CATGATCTCCCGGACCCCTGAGGTCACGTGCGTGG
TGGTGGACGTGAGCCAGGAAGACCCCGAGGTCCAG
TTCAACTGGTACGTGGATGGCGTGGAGGTGCATAA
TGCCAAGACAAAGCCGCGGGAGGAGCAGTTCAACA
GCACGTACCGTGTGGTCAGCGTCCTCACCGTCCTG
CACCAGGACTGGCTGAACGGCAAGGAGTACAAGTG
CAAGGTCTCCAACAAAGGCCTCCCGTCCTCCATCG
AGAAAACCATCTCCAAAGCCAAAGGGCAGCCCCGA
GAGCCACAGGTGTACACCCTGCCCCCATCCCAGGA
GGAGATGACCAAGAACCAGGTCAGCCTGACCTGCC
TGGTCAAAGGCTTCTACCCCAGCGACATCGCCGTG
GAGTGGGAAAGCAATGGGCAGCCGGAGAACAACTA
CAAGACCACGCCTCCCGTGCTGGACTCCGACGGCT
CCTTCTTCCTCTACAGCAGGCTAACCGTGGACAAG
AGCAGGTGGCAGGAGGGGAATGTCTTCTCATGCTC
CGTGATGCATGAGGCTCTGCACAACCACTAGACAC
AGAAGAGCCTCTCCCTGTCTCTGGGT
Antibody 1 Light chain DNA sequence:
SEQ ID NO: 6
GAAATTGTGTTGACACAGTCTCCAGCCACCCTGTC
TTTGTCTCCAGGGGAAAGAGCCACCCTCTCCTGCA
GGGCCAGTCAAAGTATCAGCAATAACCTACACTGG
TACCAACAGAAACCTGGCCAGGCTCCCAGGCTCCT
CATCTATTATACTTCCCGGTCCGTCTCTGGCATCC
CAGCCAGGTTCAGTGGCAGTGGGTCTGGGACAGAC
TTCACTCTCACCATCAGCAGCCTAGAGCCTGAAGA
TTTTGCAGTTTATTACTGTGGACAGAATAACGAGT
GGCCTGAGGTGTTCGGCGGAGGGACCAAGGTGGAG
ATCAAACGAACTGTGGCTGCACCATCTGTCTTCAT
CTTCCCGCCATCTGATGAGCAGTTGAAATCTGGAA
CTGCCTCTGTTGTGTGCCTGCTGAATAACTTCTAT
CCCAGAGAGGCCAAAGTACAGTGGAAGGTGGATAA
CGCCCTCCAATCGGGTAACTCCCAGGAGAGTGTCA
CAGAGCAGGACAGCAAGGACAGCACCTACAGCCTC
AGCAGCACCCTGACGCTGAGCAAAGCAGACTAGGA
GAAACACAAAGTCTACGCCTGCGAAGTCACCCATC
AGGGCCTGAGCTCGCCCGTCACAAAGAGCTTCAAC
AGGGGAGAGTGC
Antibody 1 LCDR1:
SEQ ID NO: 7
RASQSISNNLH
Antibody 1 LCDR2:
SEQ ID NO: 8
YTSRSVS
Antibody 1 LCDR3:
SEQ ID NO: 9
GQNNEWPEV
Antibody 1 HCDR1:
SEQ ID NO: 10
GYEFTSYWIH
Antibody 1 HCDR2:
SEQ ID NO: 11
NISPNSGSANYNEKFKS
Antibody 1HCDR3:
SEQ ID NO: 12
EGPYSYYPSREYYGSDL
Human Gro-alpha (CXCL1):
SEQ ID NO: 13
ASVATELRCQCLQTLQGIHPKNIQSVNVKSPGPHC
AQTEVIATLKNGRKACLNPASPIVKKIIEKMLNSD
KSN
Human Gro-beta (CXCL2):
SEQ ID NO: 14
APLATELRCQCLQTLQGIHLKNIQSVKVKSPGPHC
AQTEVIATLKNGQKACLNPASPMVKKIIEKMLKNG
KSN
Human Gro-gamma (CXCL3):
SEQ ID NO: 15
ASVVTELRCQCLQTLQGIHLKNIQSVNVRSPGPHC
AQTEVIATLKNGKKACLNPASPMVQKIIEKILNKG
STN
Human ENA-78 (CXCL5):
SEQ ID NO: 16
AAVLRELRCVCLQTTQGVHPKMISNLQVFAIGPQC
SKVEVVASLKNGKEICLDPEAPFLKKVIQKILDGG
NKEN
Human GCP-2 (CXCL6):
SEQ ID NO: 17
VSAVLTELRCTCLRVTLRVNPKTIGKLQVFPAGPQ
CSKVEVVASLKNGKQVCLDPEAPFLKKVIQKILDS
GNKKN
Human NAP-2 (CXCL7):
SEQ ID NO: 18
AELRCMCIKTTSGIHPKNIQSLEVIGKGTHCNQVE
VIATLKDGRKICLDPDAPRIKKIVQKKLAGDESAD
Human IL-8 (CXCL8):
SEQ ID NO: 19
SAKELRCQCIKTYSKPFHPKFIKELRVIESGPHCA
NTEIIVKLSDGRELCLDPKENWVQRVVEKFLKRAE
NS

1. A method of preventing acute respiratory distress syndrome (ARDS) in a patient in need thereof, comprising administering to the patient a therapeutically effective amount of an antibody that binds human growth-regulated oncogene (“Gro”)-alpha, human Gro-beta, human Gro-gamma, human epithelial neutrophil activating peptide-78, human granulocyte chemotactic protein-2, human neutrophil activating protein-2, and human interleukin-8, wherein the antibody comprises light chain complementarity determining regions (“LCDR”) LCDR1, LCDR2, LCDR3, and heavy chain complementarity determining regions (“HCDR”) HCDR1, HCDR2, HCDR3, wherein LCDR1 comprises SEQ ID NO: 7, LCDR2 comprises SEQ ID NO: 8, LCDR3 comprises SEQ ID NO: 9, HCDR1 comprises SEQ ID NO: 10, HCDR2 comprises SEQ ID NO: 11, and HCDR3 comprises SEQ ID NO: 12.

2. The method of claim 1, wherein the patient is at risk of developing ARDS.

3. The method of claim 1, wherein the patient has a respiratory insult.

4. The method of claim 3, wherein the respiratory insult is a respiratory disease.

5. The method of claim 3, wherein the respiratory insult is a respiratory injury.

6. A method of treating acute respiratory distress syndrome (ARDS) in a patient in need thereof, comprising administering to the patient a therapeutically effective amount of an antibody that binds human growth-regulated oncogene (“Gro”)-alpha, human Gro-beta, human Gro-gamma, human epithelial neutrophil activating peptide-78, human granulocyte chemotactic protein-2, human neutrophil activating protein-2, and human interleukin-8, wherein the antibody comprises light chain complementarity determining regions (“LCDR”) LCDR1, LCDR2, LCDR3, and heavy chain complementarity determining regions (“HCDR”) HCDR1, HCDR2, HCDR3, wherein LCDR1 comprises SEQ ID NO: 7, LCDR2 comprises SEQ ID NO: 8, LCDR3 comprises SEQ ID NO: 9, HCDR1 comprises SEQ ID NO: 10, HCDR2 comprises SEQ ID NO: 11, and HCDR3 comprises SEQ ID NO: 12.

7. The method of claim 6, wherein the patient is diagnosed as having mild ARDS.

8. The method of claim 6, wherein the patient is diagnosed as having moderate ARDS.

9. The method of claim 6, wherein the patient is diagnosed as having severe ARDS.

10. The method of claim 6, wherein the patient is diagnosed as having one of mild, moderate or severe ARDS according to the Berlin definition.

11. The method of claim 6, wherein the patient has a viral infection.

12. The method of claim 11, wherein the viral infection is a coronavirus infection.

13. The method of claim 12, wherein the coronavirus is SARS-CoV-2.

14. The method of claim 6, wherein the patient has pneumonia, asthma, COPD, interstitial lung disease and/or pulmonary fibrosis.

15. The method of claim 6, wherein the antibody comprises a heavy chain variable region comprising the amino acid sequence of SEQ ID NO: 2 and a light chain variable region comprising the amino acid sequence of SEQ ID NO: 4.

16. The method of claim 6, wherein the antibody comprises a heavy chain having the amino acid sequence of SEQ ID NO: 1 and a light chain having the amino acid sequence of SEQ ID NO: 3.

17. The method of claim 6, wherein the antibody is administered at a dose of about 100 mg to about 1200 mg intravenously.

18. The method of claim 6, wherein the antibody is administered at a dose of about 150 mg to about 1800 mg subcutaneously.

19. The method of claim 6, wherein the antibody is administered at about 1200 mg intravenously for two to three doses.

20.-29. (canceled)

30. The method of claim 1, wherein the patient has a viral infection.

31. The method of claim 30, wherein the viral infection is a coronavirus infection.

32. The method of claim 31, wherein the coronavirus is SARS-CoV-2.

33. The method of claim 1, wherein the patient has pneumonia, asthma, COPD, interstitial lung disease and/or pulmonary fibrosis.

34. The method of claim 1, wherein the antibody comprises a heavy chain variable region comprising the amino acid sequence of SEQ ID NO: 2 and a light chain variable region comprising the amino acid sequence of SEQ ID NO: 4.

35. The method of claim 1, wherein the antibody comprises a heavy chain having the amino acid sequence of SEQ ID NO: 1 and a light chain having the amino acid sequence of SEQ ID NO: 3.

36. The method of claim 1, wherein the antibody is administered at a dose of about 100 mg to about 1200 mg intravenously.

37. The method of claim 1, wherein the antibody is administered at a dose of about 150 mg to about 1800 mg subcutaneously.

38. The method of claim 1, wherein the antibody is administered at about 1200 mg intravenously for two to three doses.

39.-40. (canceled)
